(19) United States
(12) Patent Application Publication
(10) Pub. No.: US 2011/0295001 A1
(43) Pub. Date: Dec. 1, 2011

(54) Method for Producing Triazine Carbamates Using Chloroformates

(75) Inventors: René Dicke, Leonding (AT); Martin Burger, Linz (AT); Andreas Endesfelder, Linz (AT); Christoph Hahn, Linz (AT); Sven Poser, Breitenbach (DE); Willy Frank, Bad Lauchstadt (DE); Manfred Arnold, Leissling (DE)

(73) Assignee: BOREALIS AGROLINZ MELAMINE GMBH, Linz (AT)

(21) Appl. No.: 13/133,488

(22) PCT Filed: Dec. 7, 2009
(86) PCT No.: PCT/EP2009/066538

§ 371 Date: Aug. 4, 2011

(30) Foreign Application Priority Data
Dec. 9, 2008 (DE) 102008061139.5

(51) Int. Cl. C07D 251/70; C07D 403/12

(52) U.S. Cl. 544/198; 544/196

(57) ABSTRACT

The present invention relates to a method for producing triazine carbamates by conversion of at least one triazine with at least one chloroformate in the presence of at least one alkaline or alkaline earth metal compound, wherein the alkaline or alkaline earth metal compound is not present in form of an alcoholate.

The present invention relates to a method for producing triazine carbamates according to claim 1.

The U.S. Pat. No. 5,084,541 describes tricarbamoyl triazines (triazine tricarbamates, melamine tricarbamates) which are synthesized starting from triazine triisocyanate by conversion with different alcohols. These always three times substituted products can be for instance used as cross linking agents. The disadvantage of this method is that the isocyanate has to be isolated as highly reactive intermediate stage. In this manner also maximal triazine-tri-carbamates can be produced.

The DE 10 2004 018 543 A1 describes also carbamate-group containing triazine derivatives which serve as cross linking agents for varnish with improved properties. No further details are given for the production of the carbamates.

The DE 10259672 describes the production of alkoxycarbonylamino triazines by converting di- and triamino triazines with cyclic carbonates, wherein however a large amount of a base is required.

U.S. Pat. No. 6,063,922 describe triazine carbamates which are formed by a conversion of melamine and acyclic organic carbonates in the presence of a strong base. Thereby, always bi- or tricarbamates are formed. A disadvantage of this process is the use of a base, which has to be added in large amounts in order to obtain sufficient conversion. The conversion of melamine with butylchloroformate in the presence of alcoholic sodium butoxide is also described. However, this conversion only provides low yields, since a number of side reactions occur.

The object of the invention was to develop a new method, which can be easily carried out, provides good yields, preferably above 90%, and avoids the above-mentioned disadvantages.

It was now surprisingly shown that the production of at least one triazine carbamate of the formula I

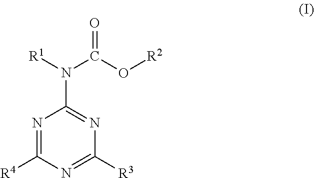

or mixtures thereof, wherein
R³ means Q¹ or a moiety of the formula R⁵—N—R⁶ bound with its central nitrogen atom to a C-atom of the triazine ring of the structure of formula (I), wherein
   Q¹ is a linear or branched C₁-C₅₀-alkyl or a cyclic substituent in form of a C₅-C₂₀-cycloalkyl, a C₅-C₂₀-aryl, C₂-C₂₀-heterocycle, C₂-C₂₀-alkenyl substituted C₂-C₂₀-heterocycle, C₁-C₅₀-alkyl substituted C₂-C₂₀-heterocycle, a C₁-C₅₀-alkyl substituted C₅-C₂₀-aryl, C₂-C₂₀-alkenyl, a C₂-C₂₀-alkenyl substituted C₅-C₂₀-aryl, C₂-C₁₂-alkinyl or an imide of cyclic saturated or unsaturated carboxylic acids, which in each case can be interrupted by one or multiple oxygen atoms, sulphur atoms, substituted and/or unsubstituted nitrogen atoms, by double bounds, siloxane groups and/or by one or multiple groups of the type —C(O)O—, —OC(O)—, —C(O)—, —NHC(O)O—, —OC(O)NH— and/or —OC(O)O—,
R⁴ means Q¹ or a moiety of the formula R⁷—N—R⁸ bound with a nitrogen atom to a C-atom of the triazine ring of the structure of formula (I),
R¹, R⁵, R⁶, R⁷ and R⁸ mean independently from each other H, Q², —CO—O—R², —CO—R⁹ or —CO—O—R¹⁰ wherein
   Q² is in each case a linear or branched C₁-C₅₀-alkyl, C₅-C₂₀-cycloalkyl, C₅-C₂₀-aryl, C₁-C₅₀-alkyl substituted C₅-C₂₀-aryl, C₂-C₂₀-heterocycles, C₂-C₂₀-alkenyl substituted C₂-C₂₀-heterocycle, C₁-C₅₀-alkyl substituted C₂-C₂₀-heterocycle, C₂-C₂₀-alkenyl, C₂-C₁₂-alkinyl or C₂-C₂₀-alkenyl substituted C₅-C₂₀-aryl, which in each case can be interrupted by one or multiple oxygen atoms, sulphur atoms, substituted and/or unsubstituted nitrogen atoms, by double bounds, siloxane groups and/or by one or multiple groups of the type —C(O)O—, —CO(O)—, —C(O)—, —NHC(O)O—, —OC(O)NH— and/or —OC(O)O—,
R² is a linear or branched C₁-C₅₀-alkyl, C₅-C₂₀-cyclo alkyl, C₅-C₂₀-aryl, C₁-C₅₀-alkyl substituted C₅-C₂₀-aryl, C₂-C₂₀-heterocycle, C₂-C₂₀-alkenyl substituted C₂-C₂₀-heterocycle, C₁-C₅₀-alkyl substituted C₂-C₂₀-heterocycle, C₂-C₁₂-alkinyl, C₂-C₂₀-alkenyl or C₂-C₂₀-alkenyl substituted C₅-C₂₀-aryl, which in each case can be interrupted by one or multiple oxygen atoms, sulphur atoms, substituted and/or unsubstituted nitrogen atoms, by double bounds, siloxane groups and/or by one or multiple groups of the type —C(O)O—, —OC(O)—, —C(O)—, —NHC(O)O—, —OC(O)NH— and/or —OC(O)O— and/or have one or multiple halogen atoms and/or nitro groups as substituents

R⁹ means a moiety of the general formula (II)

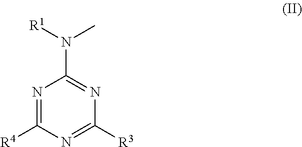

R¹⁹ means a moiety of the general formula (III)

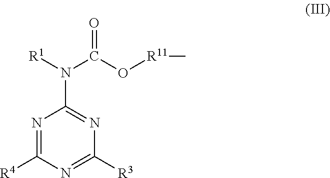

wherein
R¹¹ is in each case a linear or branched C₁-C₅₀-alkyl, C₅-C₂₀-cycloalkyl, C₅-C₂₀-aryl, C₁-C₅₀-alkyl substituted C₅-C₂₀-aryl, C₂-C₂₀-heterocycle, C₂-C₂₀-alkenyl substituted C₂-C₂₀-heterocycle, C₁-C₅₀-alkyl substituted C₂-C₂₀-heterocycle, C₂-C₂₀-alkenyl, C₂-C₁₂-alkinyl, or C₂-C₂₀-alkenyl substituted C₅-C₂₀-aryl, which can be interrupted in each case by one or multiple oxygen atoms, sulphur atoms, substituted and/or unsubstituted nitrogen atoms, by double bounds, siloxane groups and/or by one or multiple groups of the type —C(O)O—, —OC(O)—, —C(O)—, —NHC(O)O—, —OC(O)NH— and/or —CO(O)O—,
   is possible by conversion of at least one triazine of the formula IV

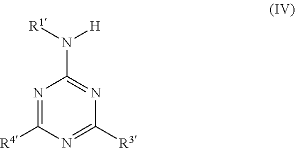

wherein R^{1′} has the meaning of R¹, R^{3′} the meaning of R³ and R^{4′} has the meaning of R4, R⁴
with at least one chloroformate of the general formula (V)

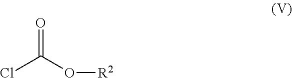

and/or the general formula (VI)

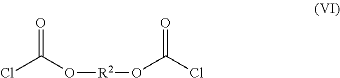

in the presence of at least one alkaline or alkaline earth metal compound, wherein the alkaline or alkaline earth metal compound is not present in form of an alcoholate.

As herein used, the term “C₁-C₅₀-alkyl” designates moieties as methyl, ethyl, n-propyl, isopropyl, n-butyl, isobutyl, s-butyl, t-butyl, amyl, t-amyl, hexyl, heptyl, octyl, nonyl, decyl, undecyl, dodecyl and longer chain moieties. Preferred C₁-C₅₀-alkyl groups are methyl, ethyl, propyl, isopropyl and butyl.

The term “C₅-C₂₀-cycloalkyl” comprises amongst others the groups cyclopropyl, cyclopentyl, cyclohexyl, cycloheptyl and higher membered rings.

The term “C₅-C₂₀-aryl” as used herein designates aromatic hydrocarbons, for example phenyl, benzyl, naphtyl or anthryl.

The term “C₂-C₂₀-heterocyclu” designates optionally substituted rings with 2-20-C-atoms, which have 1 to 4 heteroatoms as oxygen, sulphur and/or nitrogen, in particular nitrogen, either alone or in combination with sulphur or oxygen ring atoms. These rings can be saturated or completely unsaturated or partially unsaturated, wherein completely saturated rings are preferred. Preferred heterocyclic rings include piperidinyl, morpholinyl, piperazinyl, 2-amino-imidazoyl, tetrahydrofurano, pyrrolo, tetrahydrothiophenyl.

The term, “C₂-C₂₀-alkenyl” designates a moiety comprising a double bound, wherein said moiety can be substituted or unsubstituted. The stereoisomery is not essential and all stereoisomers can be used for a respectively substituted alkenyl.

The term “C₂-C₁₂-alkinyl” as used herein designates the moiety of the formula C₂-C₁₂-C≡C—. Examples for C₂-C₁₂-alkinyles include: ethinyl, propinyl or propargyl, 2-butinyl, 2-pentinyl, 3-pentinyl, 2-hexinyl, 3-hexinyl, 4-hexinyl, 2-heptinyl, 3-heptinyl, 4-heptinyl, 5-heptinyl, as well as octinyl, noninyl, decinyl, undecinyl, dodecinyl, as well as di- and tri-ine of straight or branched alkyl chains. Such alkinyl moieties are preferred in which the triple bound is terminal.

The term “substituted” when using with “alkyl”, “alkenyl” etc. designates the substitution of one or multiple atoms, usually H-atoms, by one or multiple of the following substituents, preferably by one or two of the following substituents: halogen, hydroxy, protected hydroxy, oxo, protected oxo, C₃-C₇-cycloalkyl, phenyl, naphtyl, amino, protected amino, monosubstituted amino, protected monosubstituted amino, disubstituted amino. Further substituents are in general conceivable. The substituted alky groups, aryl groups, alkenyl groups can once or multiply be substituted and preferably once or twice with the same or different substituents.

In the method according to the invention also any mixtures of different triazines of the formula IV can be used and reacted as starting compounds. Thus, the corresponding mixtures of the triazine carbamates (I) are obtained.

The ongoing reaction is exemplarily illustrated in the following reaction equation:

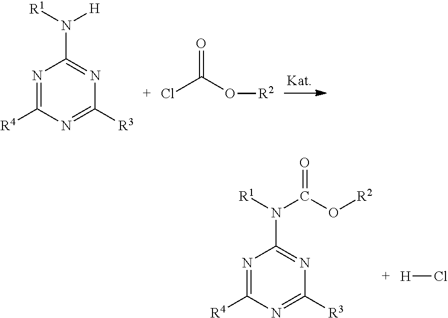

The used alkaline or alkaline earth metal compound can also be ideally used besides its effect as activator in order to bind the HCl being released in form of an alkaline or alkaline earth metal chloride.

In the method according to the invention advantageously alkaline or alkaline earth metal compounds from a group comprising NaHCO₃, KHCO₃, Na₂CO₃, K₂CO₃, MgCO₃, CaCO₃, Na₃PO₄, Na₂HPO₄, Na-acetate, disodium oxalate, butyl lithium, methyl lithium, phenyl lithium, methyl sodium, butyl sodium, phenyl sodium, methylmagnesium bromide, LiAlH₄, sodium amide are used, wherein preferably NaCO₃, Na₂CO₃ and butyllithium are used.

The alkaline and alkaline earth compounds are used in an amount of 0.05 to 1.2 mol equivalents in respect to the NH-groups to be converted. Thereby, preferably 0.5 to 1.2 mol equivalents of alkaline and alkaline earth compounds, in particular preferably 0.8 to 1.2 mol equivalents of alkaline and alkaline earth compounds are used.

The application of alkaline or alkaline earth metal compounds in stoichiometric amounts or in a slight excess in respect to the NH groups to be converted guarantees a rapid progress of the reaction.

Optionally, a further compound is added to the reaction mixture, which binds the released HCl and accelerates thus the reaction progress. Suitable compounds herefore are for instance triethylamine, diethylamine, butylamine, dibutylamine. It is thereby of an advantage if the further compound is used in an amount of 0.5 to 1.5 mol equivalents per NH group to be converted.

In the method according to the invention 0.7 to 10 mol to 10.0 mol, preferably 0.9 to 7.0 mol chloroformate of the formula V or the formula VI are used per mol equivalent NH-groups in the triazine of the formula IV.

The method is being advantageously carried out in substance, wherein the chloroformate of the formula V or the formula VI acts also as solvent, and/or in another suitable solvent. The reaction is carried out preferably in solution.

Suitable solvents for carrying out the methods according to the invention are tetrahydrofuran, diethylether, dimethoxymethane, dimethoxyethane, diethoxymethane, diethoxyethane, ethylenglycoldimethylether, ethylenglycoldiethylether, ethylenglycoldibutylether, diethylenglycoldiethylether, dioxane, acetone, methylenechloride, chloroform, benzene, toluene, xylene, mesitylene, cumen, chlorobenzene, pentane, hexane, cyclohexane, heptane, octane, acetonitril, methylacetate, ethylacetate, methylbenzoate, N,N-dimethylformamid, N,N-dimethylacetamid, N-methylpyrrolidone, 1,3-dimethylimidazolidinon as well as ionic liquids known as solvents for the person skilled in the art.

The method according to the invention is usually carried out at a temperature of 0 to 200° C. It has advantageously been proven, if a temperature profile is run during the reaction.

The reaction is ideally started at low temperatures and is then increased slowly to the desired final temperature. The reaction is advantageously started at temperatures between 0° C. and 20° C. (room temperature) and is then increased to the selected final temperature between 20° C. and 200° C. Higher reaction temperatures allow thereby a double substitution at an NH₂— group of the used triazine.

Advantageously the method according to the invention is carried out such that the triazine compound IV is provided in a suitable solvent, is mixed with the alkaline or alkaline earth metal compound at low temperature and subsequently the chloroformate is added. Then, an increase of the temperature occurs advantageously for completing the reaction. The isolation and purification of the reaction product is carried out according to methods known to the person skilled in the art.

Advantageously as chloroformates methylchloroformate, butylchloroformate, phenylchloroformate, benzylchloroformate, menthylchloroformate, 1-chloroethylchloroformate, 1-naphthylchloroformate, 2-chloroethylchloroformate, 2-chlorobenzylchloroformate, 2-chlorophenylchloroformate, 2-ethylhexylchloroformate, 2-fluorethylchloroformate, 2-methoxyethylchloroformate, 2-methoxyphenylchloroformate, 2-nitrophenylchloroformate, 2-chloropropylchloroformate, 4-chlorobutylchloroformate, allylchloroformate, cetylchloroformate, ethylchloroformate, ethylen-bis(chloroformate), hexylchloroformate, isobutylchloroformate, isopropenylchloroformate, neopentylchloroformate, octylchloroformate, tolylchloroformate, propargylchloroformate, propylchloroformate, vinylchloroformate, 1,4-butandiol-bis(chloroformate), 2-butyn-1-ylchloroformate, 3-butyn-1-ylchloroformate, bisphenol-A-bis(chloroformate), bisphenol-Z-bis(chloroformate), triethylenglycol-bis(chloroformate), 1,4-phenylen-bis(chloroformate) are used.

Preferably used chloroformates are methylchloroformate, butychloroformate, phenylchloroformate, allylchloroformate, ethylen-bis(chloroformate), isobutylchloroformate, vinylchloroformate, 1,4-butandiol-bis(chloroformate), bisphenol-A-bis(chloroformate) chloropropylchloroformate and propargylchloroformate.

The triazine carbamates I formed by the method according to the invention can advantageously react by splitting off the hydroxyl compound R²—OH with a further triazine compound IV so that polynuclear triazine compounds, in particular 2 to 20 nuclear triazine compounds are produced. This reaction corresponds in principle to a transesterification or a transamidation. Thus, at the end a urea compound is built up from the carbamate compound, whereat two triazine nuclei are linked with each other.

Since this reaction can occur also multiple times in the presence of the suitable groups the assembly of polynuclear triazine complexes is thus possible. The frequency of this reaction can be selectively controlled by the selection of the reaction conditions.

Also, by variation of the dosage sequence and the dosage velocity it is possible to control the frequency of the formation of polynuclear compounds. Thus, the chloroformate is usually added to a mixture of triazine and alkaline or alkaline earth metal compound. If this addition occurs fast then almost no polynuclear compounds are formed. If in contrast the addition occurs slowly, then the above described reaction is enforced and polynuclear compounds are formed.

The triazine carbamates obtainable after a method according to the invention are valuable raw materials, for instance for the varnish industry. The triazine carbamates can be used as cross linking agents as well as in the set up of binders, for instance in polyester, polyurethane or epoxy-based varnishes.

The invention is explained in the following by the means of multiple examples.

### EXAMPLE 1

In a reactor equipped with stirrer, reflux condenser and nitrogen inlet 100 g N,N′,N″-trimethylmelamine, 200 g sodiumhydrogencarbonate and 1.0 l methylchloroformate are provided in 1.5 l dry dichloromethane and heated slowly until reflux. During the whole reaction a slight nitrogen flow is maintained in the system. After 7.5 h it is being cooled to room temperature and 200 g sodiumsulfate are added. Subsequently, the mixture is filtrated and the filtrate is reduced in a rotary evaporator and dried overnight in vacuum. 192 g of a colourless solid, which consists of 96.8% of N,N′,N″-tri-(methylcarbamoyl)-N,N′,N″-trimethylmelamine.

### EXAMPLE 2

1.0 g N,N′,N″-trimethylmelamine and 2.0 g sodium hydrogencarbonate are provided in a flask in 10 ml dry THF and subsequently 5 ml methylchloroformate are added at room temperature and stirred. 2.0 g sodium sulfate are added after 40 h, filtrated and the filtrate is reduced and dried in vacuum. 1.6 g of a white solid is obtained, which consists of 18% of N,N′-di-(methylcarbamoyl)-N,N′,N″-trimethylmelamine and of 81% of N,N′,N″-tri-(methylcarbamoyl)-N,N′,N″-trimethylmelannine.

### EXAMPLE 3

1.0 g N,N′,N″-trimethylmelamine and 2.0 g sodium hydrogencarbonate are provided in a flask in 15 ml dry ethylacetate and subsequently 5 ml methylchloroformate are added at room temperature and stirred. 2.0 g sodium sulfate are added after 15 h, filtrated and the filtrate is reduced and dried in vacuum. 1.3 g of a white solid is obtained, which consists of 23% of N-(methylcarbamoyl)-N,N′,N″-trimethylmelamine, 32% of N,N′-di-(methylcarbamoyl)-N,N′,N″-trimethylmelamine and of 43% of N,N′,N″-tri-(methylcarbamoyl)-N,N′,N″-trimethylmelamine.

### EXAMPLE 4

In a 250 ml three-necked flask equipped with stirrer, reflux condenser and nitrogen inlet 5.0 g (29.7 mmol) N,N′,N″-trimethylmelamine and 10.0 g (119 mmol) sodium hydrogencarbonate are provided in 75 ml dry methylenechloride and are heated to 30° C. Subsequently, 20 ml (259 mmol) methylchloroformate are added dropwise. Thereupon it is stirred at 30° C. and the increase of formed N,N′,N″-tri-(methylcarbamoyl)-N,N′,N″-trimethylmelamine is determined by the means of GC/FID. The values can be taken from the following table 1.

TABLE 1
Formation of N,N′,N″-tri-(methylcarbamoyl)-N,N′,N″-
trimethylmelamine in dependency on the reaction time.

| | Product content/ |
|---|---|
| Reaction time/h | m % |
| 3 | 18.5 |
| 20 | 54 |
| 25.5 | 69 |
| 44 | 76 |

### EXAMPLE 5

In a 250 ml three-neck flask equipped with stirrer, reflux condenser and nitrogen inlet 5.0 g (29.7 mmol) N,N′,N″-trimethylmelamine and 10.0 g (119 mmol) sodium hydrogencarbonate are provided in 75 ml dry methylenechloride. Subsequently, 20 ml (259 mmol) methylchloroformate are added dropwise at room temperature. Thereupon, it is heated to 40° C. After 3 h it is cooled to room temperature and 10 g of sodium sulfate are added. Subsequently, the mixture is filtrated and the filtrate is reduced at the rotary evaporator and dried overnight in vacuum. 16.3 g of a colourless solid is obtained, which consists of 7% N-(methylcarbamoyl)-N,N′,N″-trimethylmelamine, 38% N,N′-di-(methylcarbamoyl)-N,N′,N″-trimethylmelamine and 55% N,N′,N″-tri-(methylcarbamoyl)-N,N′,N″-trimethylmelamine.

### EXAMPLE 6

In a 250 ml three-neck flask equipped with stirrer, reflux condenser and nitrogen inlet 5.0 g (29.7 mmol) N,N′,N″-trimethylmelamine and 10.0 g (119 mmol) sodium hydrogencarbonate are provided in 75 ml dry methylenechloride. Subsequently, 50 ml (648 mmol) methylchloroformate are added dropwise at room temperature. Thereupon it is heated to 40° C. After 3 hours it is cooled to room temperature and 10 g sodium sulfate are added. Subsequently, the mixture is filtrated and the filtrate is reduced in a rotary evaporator and dried overnight in vacuum. 18.4 g of a colourless solid are obtained, which consists of 21% N,N′-di-(methylcarbamoyl)-N,N′,N″-trimethylmelamine and 79% N,N′,N″-tri-(methyl-carbamoyl)-N,N′,N″-trimethylmelamine.

### EXAMPLE 7

In a 500 ml round flask 15.0 g (0.09 mol) N,N′,N″-trimethylmelamine are dissolved in 240 ml dioxane and cooled to 10° C. Then, 157 ml (0.283 mol) n-butyllithium (1.6 molar in hexane) are added dropwise at 10° C. It is stirred for an hour. Subsequently, 30 ml (0.389 mol) methylchloroformate in 30 ml dioxane are rapidly added dropwise by intensive cooling in a time range of 15 min. After completed addition it is being allowed to warm up to room temperature and it is being stirred for further 20 h. Then, the reaction mixture is poured into 600 ml hydrochloric acid (pH=2), wherein a clear solution is formed. A pH value of 6 is adjusted using NaOH-solution and is stirred for ca. 1 hour in order to decompose excess methylchloroformate. Then the solution is largely reduced and the precipitate formed thereby is filtered. After drying the precipitate 27.3 g of a white solid are obtained which consists of 1.8% N,N′-di-(methylcarbamoyl)-N,N′,N″-trimethylmelamine, 79.5% N,N′,N″-tri-(methylcarbamoyl)-N,N′,N″-trimethylmelamine, 13.4% of the binuclear species of the formula (VII) shown below and 4.2% of analogue three- and four-nuclear species.

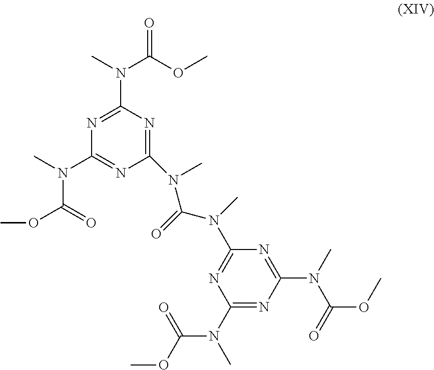

### EXAMPLE 8

In a 500 ml round flask 15.0 g (0.09 mol) N,N′,N″-trimethylmelamine are dissolved in 240 ml dioxane and cooled to 10° C. Then 157 ml (0.283 mol) n-butyllithium (1.6 molar in hexane) are added dropwise at 10° C. It is being stirred for an hour. Subsequently, 30 ml (0.389 mol) methylchloroformate in 30 ml dioxane are slowly added dropwise via a time period of 45 min. After completed addition it is being warmed up to room temperature and is being stirred for further 20 h. Then the reaction mixture is poured into 600 ml hydrochloric acid (pH=2), wherein a clear solution is formed. A pH value of 6 is adjusted with NaOH solution and stirred for ca. 1 h in order to decompose excess methylchloroformate. Then, the solution is largely reduced and the precipitate formed thereby is filtrated. After drying the precipitate 29.2 g of a white solid are obtained, which consists of 0.1% N,N′-di-(methylcarbamoyl)-N,N′,N″-trimethylmelamine, 17.1% N,N′,N″-tri-(methylcarbamoyl)-N,N′,N″-trimethylmelamine, 17.9% of the binuclear species of the formula (XIV) as illustrated and 60.3% of analogue three- and four-nuclear species.

### EXAMPLE 9

In a 250 ml round flask 5.0 g (0.03 mol) N,N′,N″-trimethylmelamine are dissolved in 80 ml THF and cooled to 0° C. Then 59 ml (0.106 mol) n-butyllithium (1.6 mol in hexane) is added dropwise. It is being stirred for an hour. Subsequently 14.5 ml (0.188 mol) methylchloroformate in 15 ml THF are added dropwise by intensive cooling. After completed addition it is being stirred for 2 h at 0° C. and is then allowed to warm up to room temperature and is being stirred for further 20 h. Then the reaction mixture is poured into 200 ml water/HCl (pH=2) wherein a clear solution is formed. A pH value of 6 is adjusted with NaOH solution and stirred for ca. 1 h, in order to decompose excess methylchloroformate. Then the solution is largely reduced and the precipitate formed thereby is filtrated. After drying the precipitate 9.7 g of a white solid are obtained, which consists of 2.3% N,N′-di-(methylcarbamoyl)-N,N′,N″-trimethylmelamine, 85.2% N,N′,N″-tri-(methylcarbamoyl)-N,N′,N″-trimethylmelamine and 12.5% of the binuclear species of the formula (XIV).

### EXAMPLE 10

In a 250 ml round flask 5.0 g (0.03 mol) N,N′,N″-trimethylmelamine are dissolved in 80 ml dioxane and cooled to 10° C. Then 84 ml (0.151 mol) n-butyllithium (1.6 molar in hexane) are added dropwise at 10° C. It is being stirred for an hour. Subsequently, 20.7 ml (0.268 mol) methylchloroformate in 20 ml dioxane are added dropwise with intensive cooling. After completed addition it is still being stirred for 2 h at 10° C. and is then allowed to warm up to room temperature and is stirred for further 20 h. Then the reaction mixture is poured into 200 ml water/HCl (pH=2), wherein a clear solution is formed. A pH value of 6 is adjusted with NaOH solution and stirred for 1 h in order to decompose excess methylchloroformate. Then the solution is largely reduced and the precipitate formed thereby is filtrated. After drying the precipitate 10.6 g of a white solid are obtained, which consists of 5.6% N,N′-di-(methylcarbamoyl)-N,N′,N″-trimethylmelamine, 78.8% N,N′,N″-tri-(methylcarbamoyl)-N,N′,N″-trimethylmelamine and 15.6% of the binuclear species VI.

### EXAMPLE 11

In a 250 ml round flask 5.0 g (0.03 mol) N,N′,N″-trimethylmelamine are dissolved in 80 ml dioxane and cooled to 10° C. Then 59 ml (0.106 mol) n-butyllithium (1.6 molar in hexane) are added dropwise at 10° C. It is being stirred for an hour. Subsequently, 14.5 ml (0.188 mol) methylchloroformate in 15 ml dioxane are added dropwise with intensive cooling. After completed addition it is still being stirred for 2 h at 10° C. and is then allowed to warm up to room temperature and is being stirred for further 20 h. Then the reaction mixture is poured into 200 ml water/HCl (pH=2), wherein a clear solution is formed. A pH value of 6 is adjusted with NaOH solution and it is stirred for 1 h in order to decompose excess methylchloroformate. Then the solution is largely reduced and the precipitate formed thereby is filtrated. After drying the precipitate 10.6 g of a white solid are obtained, which consists of 2.8% N,N′-di-(methylcarbamoyl)-N,N′,N″-trimethylmelamine, 87.5% N,N′,N″-tri-(methylcarbamoyl)-N,N′,N″-trimethylmelamine and 9.7% of the binuclear species VI.

### EXAMPLE 12

In a 250 ml round flask 4.62 g (0.03 mol) N,N′-dimethylmelamine are dissolved in 80 ml dioxane and cooled to 10° C. Then 59 ml (0.106 mol) n-butyllithium (1.6 molar in hexane) are added dropwise at 10° C. It is being stirred for an hour. Subsequently, 5.1 ml (0.066 mol) methylchloroformate in 10 ml dioxane are added dropwise with intensive cooling. After completed addition it is still being stirred for 2 h at 10° C. and is then allowed to warm up to room temperature and is being stirred for further 20 h. Then the reaction mixture is poured into 200 ml water/HCl (pH=2), wherein a clear solution is formed. A pH value of 6 is adjusted with NaOH solution and stirred ca. for 1 h in order to decompose excess methylchloroformate. Then the solution is largely reduced and the precipitate formed thereby is filtrated. After drying the precipitate 6.7 g of a white solid are obtained, which consists of 62.4% N-(methylcarbamoyl)-N,N′-dimethylmelamine, 23.9% N,N′-di-(methylcarbamoyl)-N,N′-dimethylmelamine and 7.3% N,N′,N″-tri-(methylcarbamoyl)-N,N′-dimethylmelamine.

### EXAMPLE 13

In a 250 ml round flask 3.78 g (0.03 mol) melamine are suspended in 80 ml dioxane and cooled to 10° C. Then 59 ml (0.106 mol) n-butyllithium (1.6 molar in hexane) are added dropwise at 10° C. It is being stirred for an hour. Subsequently, 8.05 ml (0.104 mol) methylchloroformate in 12 ml dioxane are added dropwise with intensive cooling. After completed addition it is still being stirred for 2 h at 10° C. and is then warmed up to room temperature and stirred for further 20 h. Over the time a clear solution is formed. Then the reaction mixture is poured into 200 ml water/HCl (pH=2), wherein a clear solution is being formed. A pH value of 6 is adjusted with NaOH solution and is stirred ca. for 1 h in order to decompose excess methylchloroformate. Then the solution is largely reduced and the precipitate formed thereby is filtrated. After drying the precipitate 3.3 g of a white solid are obtained, which consists of 69% N-(methylcarbamoyl)-melamine, 25.7% N,N′-di-(methylcarbamoyl)-melamine and 5.3% N,N′,N″-tri-(methylcarbamoyl)-melamine.

### EXAMPLE 14

In a 250 ml round flask 5.0 g (0.03 mol) N,N′,N″-trimethylmelamine are dissolved in 80 ml dioxane and cooled to 10° C. Then 59 ml (0.106 mol) n-butyllithium (1.6 molar in hexane) are added dropwise at 10° C. It is being stirred for an hour. Subsequently, 23.9 ml (0.188 mol) butylchloroformate in 25 ml dioxane are added dropwise with intensive cooling. After completed addition it is still being stirred for 2 h at 10° C. and is then warmed up to room temperature and stirred for further 20 h. Then the reaction mixture is poured into 200 ml water/HCl (pH=2), wherein two phases are formed. A pH value of 6 is adjusted with NaOH solution and is stirred ca. for 1 h in order to decompose excess butylchloroformate. Then the solution is extracted 3× with methylenechloride and the organic phase is completely reduced. 15.8 g of a clear solution are obtained, which consist of 8.4% N,N′-di-(butylcarbamoyl)-N,N′,N″-trimethylmelamine and 90.4% N,N′,N″-tri-(butylcarbamoyl)-N,N′,N″-trimethylmelamine.

### EXAMPLE 15

In a 250 ml round flask 5.0 g (0.03 mol) N,N′,N″-trimethylmelamine are dissolved in 80 ml dioxane and cooled to 10° C. Then 59 ml (0.106 mol) n-butyllithium (1.6 molar in hexane) are added dropwise at 10° C. It is being stirred for an hour. Subsequently, 23.2 g (0.108 mol) 1,4-butanediol-bis(chloroformate) in 25 ml dioxane are added dropwise with intensive cooling. After completed addition it is still being stirred for 2 h at 10° C. and is then warmed to room temperature and stirred for further 20 h. Then the reaction mixture is poured into 200 ml water/HCl (pH=2), wherein a clear solution is being formed. A pH value of 6 is adjusted with NaOH solution and stirred for ca. 1 h in order to decompose excess butylchloroformate. Then the solution is largely reduced and the precipitate formed thereby is filtrated. After drying the precipitate 15.8 g of a white solid are obtained, which consists of 74.7% of the binuclear species according to Figure XV and 18.4% of corresponding three-nuclear species as well as 6.2% of four-nuclear species.

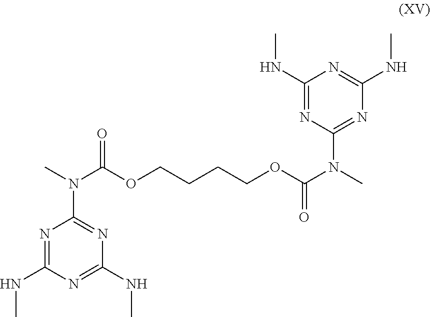

### EXAMPLE 16

In a 250 ml round flask 5.0 g (0.027 mol) N,N,N′,N″-tetramethylmelamine are dissolved in 80 ml dioxane and cooled to 10° C. Then 59 ml (0.106 mol) n-butyllithium (1.6 molar in hexane) are added dropwise at 10° C. It is being stirred for an hour. Subsequently, 15.4 ml (0.20 mol) methylchloroformate in 20 ml dioxane are added dropwise with intensive cooling. After completed addition it is still being stirred for 2 h at 10° C. and is then warmed up to room temperature and stirred for further 20 h. Then the reaction mixture is poured into 200 ml water/HCl (pH=2), wherein a clear solution is formed. A pH value of 6 is adjusted with NaOH solution and is stirred ca. 1 h in order to decompose excess methylchloroformate. Then the solution is largely reduced and the precipitate formed thereby is filtrated. After drying the precipitate 8.4 g of a white solid are obtained, which consists of 18.4% of N′-(methylcarbamoyl)-N,N,N′,N″-tetramethylmelamine and 80.6% N′,N″-di-(methylcarbamoyl)-N,N,N′,N″-tetramethylmelamine.

### EXAMPLE 17

In a 250 ml round flask 4.62 g (0.03 mol) N,N-dimethylmelamine are dissolved in 80 ml dioxane and cooled to 10° C. Then 59 ml (0.106 mol) n-butyllithium (1.6 molar in hexane) are added dropwise at 10° C. It is being stirred for an hour. Subsequently, 5.1 ml (0.066 mol) methylchloroformate in 10 ml dioxane are added dropwise with intensive cooling. After completed addition it is still being stirred for 2 h at 10° C. and is then warmed up to room temperature and stirred for further 20 h. Then the reaction mixture is poured into 200 ml water/HCl (pH=2), wherein a clear solution is formed. A pH value of 6 is adjusted with NaOH solution and is stirred for ca. 1 h in order to decompose excess methylchloroformate. Then the solution is largely reduced and the precipitate formed thereby is filtrated. After drying the precipitate 6.9 g of a white solid are obtained, which consists of 40.4% N′-(methylcarbamoyl)-N,N-dimethylmelamine, 47.8% N′,N″-di-(methylcarbamoyl)-N,N-dimethylmelamine and 10.4% N′,N′,N″-tri-(methylcarbamoyl)-N,N-dimethylmelamine.

### EXAMPLE 18

In a 250 ml round flask 6.3 g (0.03 mol) succinimidomelamine are dissolved in 80 ml dioxane and cooled to 10° C. Then 59 ml (0.106 mol) n-butyllithium (1.6 molar in hexane) are added dropwise at 10° C. It is stirred for an hour. Subsequently, 5.1 ml (0.066 mol) methylchloroformate in 10 ml dioxane are added dropwise with intensive cooling. After completed addition it is still being stirred for 2 h at 10° C. and is then warmed up to room temperature and stirred for further 20 h. Then the reaction mixture is poured into 200 ml water/HCl (pH=2), wherein a clear solution is formed. A pH value of 6 is adjusted with NaOH solution and is stirred for ca. 1 h in order to decompose excess methylchloroformate. Then the solution is largely reduced and the precipitate formed thereby is filtrated. After drying the precipitate 7.9 g of a white solid are obtained, which consists of 28.2% N′-(methylcarbamoyl)-succinimidomelamine, 65.4% N′,N″-di-(methylcarbamoyl)-succinimidomelamine and 5.9% N′,N′N″-tri-(methylcarbamoyl)-succinimidomelamine.

1-12. (canceled)

13. A method for producing triazine carbamate of formula I 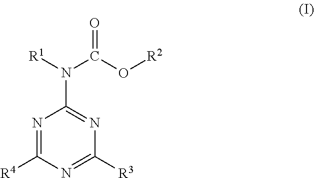
or mixtures thereof, wherein
R³ means a moiety of the formula R⁵—N—R⁶ bound with its central nitrogen atom to a C-atom of the triazine ring of the structure of formula (I),
R⁴ means a moiety of the formula R⁷—N—R⁸ bound with the nitrogen atom to a C-atom of the triazine ring of the structure of formula (I),
R⁶ and R⁸ mean independently from each other H, Q², —CO—O—R², —CO—R⁹ or —CO—O—R¹⁰ and
R¹, R⁵ and R⁷ mean independently from each other Q², —CO—O—R², —CO—R⁹ or —CO—O—R¹⁰ wherein
Q is in each case a linear or branched C₁-C₅₀-alkyl, C₅-C₂₀-cycloalkyl, C₅-C₂₀-aryl, C₁-C₅₀-alkyl substituted C₅-C₂₀-amyl, C₂-C₂₀-heterocycle, C₂-C₂₀-alkenyl substituted C₂-C₂₀-heterocycle, C₁-C₅₀-alkyl substituted C₂-C₂₀-heterocycle, C₂-C₂₀-alkenyl, C₂-C₁₂-alkinyl or C₂-C₂₀-alkenyl substituted C₅-C₂₀-aryl, which in each case can be interrupted by one or multiple oxygen atoms, sulphur atoms, substituted and/or unsubstituted nitrogen atoms, by double bounds, siloxane groups and/or by one or multiple groups of the type —C(O)O—, —OC(O)—, —NHC(O)O—, —OC(O)NH— and/or —OC(O)O—,
R² is a linear or branched C₁-C₅₀-alkyl, C₅-C₂₀-cyclo alkyl, C₁-C₅₀-aryl, substituted C₅-C₂₀-aryl, C₂-C₂₀-heterocycle, C₂-C₂₀-alkenyl substituted C₂-C₂₀-heterocycle, C₁-C₅₀-alkyl substituted C₂-C₂₀-heterocycle, C₂-C₁₂-alkinyl, C₂-C₂₀-alkenyl or C₂-C₂₀-alkenyl substituted C₅-C₂₀-aryl, which in each case can be interrupted by one or multiple oxygen atoms, sulphur atoms, substituted and/or unsubstituted nitrogen atoms, by double bounds, siloxane groups and/or by one or multiple groups of the type —C(O)O—, —OC(O)—, —C(O)—, —NHC(O)O—, —OC(O)NH— and/or —OC(O)O— and/or have one or multiple halogen atoms and/or nitro groups as substituents
R⁹ means a moiety of the general formula (II) 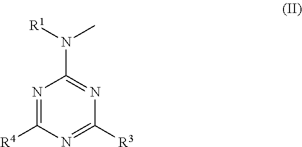
R¹⁰ means a moiety of the general formula (III) 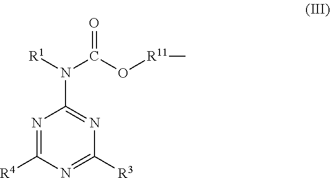
wherein
R¹¹ is in each case a linear or branched C₁-C₅₀-alkyl, C₅-C₅₀-cycloalkyl, C₅-C₂₀-aryl, C₁-C₅₀-alkyl substituted C₅-C₂₀-aryl, C₂-C₂₀-heterocycle, C₂-C₂₀-alkenyl substituted C₂-C₂₀-heterocycle, C₁-C₅₀-alkyl substituted C₂-C₂₀-heterocycle, C₂-C₂₀-alkenyl, C₂-C₁₂-alkinyl, or C₂-C₂₀-alkenyl substituted C₅-C₂₀-aryl, which can be interrupted in each case by one or multiple oxygen atoms, sulphur atoms, substituted and/or unsubstituted nitrogen atoms, by double bounds, siloxane groups and/or by one or multiple groups of the type —C(O)O—, —OC(O)—, —C(O)—, —NHC(O)O—, —OC(O)NH— and/or —OC(O)O—,
and wherein
the conversion of at least one triazine of the formula IV 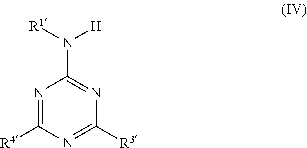
Wherein R^{1′} has the meaning of R¹, R^{3′} the meaning of R³ and R^{4′} has the meaning of R4, R⁴
with at least one chloroformat of the general formula (V) 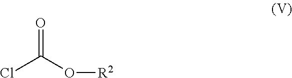
and/or the general formula (VI) 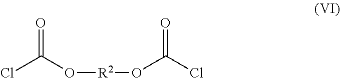
in the presence of at least one alkaline or alkaline earth metal compound from the group comprising NaHCO₃, KHCO₃, NA₂CO₃, K₂CO₃, MgCO₃, CaCO₃, Na₃PO₄, Na₂HPO₄, Na-acetate, disodium oxalate, butyl lithium, methyl lithium, phenyl lithium, methyl sodium, butyl sodium, phenyl sodium, methylmagnesium bromide, LiAlH₄, sodium amide is used, wherein the alkaline or alkaline earth metal compound is not present in form of an alcoholate.

14. The method according to claim 13, wherein as chloroformate methylchloroformate, butylchloroformate, phenylchloroformate, benzylchloroformate, menthylchloroformate, 1-chloroethylchloroformate, 1-naphthylchloroformate, 2-chloroethylchloroformate, 2-chlorobenzylchloroformate, 2-chlorophenylchloroformate, 2-ethylhexylchloroformate, 2-fluorethylchloroformate, 2-methoxyethylchloroformate, 2-methoxyphenylchloroformate, 2-nitrophenylchloroformate, 2-chloropropylchloroformate, 4-chlorobutylchloroformate, allylchloroformate, cetylchloroformate, ethylchloroformate, ethylen-bis(chloroformate), hexylchloroformate, isobutylchloroformate, isopropenylchloroformate, neopentylchloroformate, octylchloroformate, tolylchloroformate, propargylchloroformate, propylchloroformatc, vinylchloroformate, 1,4-butandiol-bis(chloroformate), 2-butyn-1-ylchloroformate, 3-butyn-1-ylchloroformate, bisphenol-A-bis(chloroformate), bisphenol-Z-bis(chloroformate), triethylenglycol-bis(chloroformate), 1,4-phenylen-bis(chloroformate) or any mixtures thereof are used.

15. The method according to claim 14, wherein as chloroformate methylchloroformate, butychloroformate, phenylchloroformate, allylchloroformate, ethylen-bis(chloroformate), isobutylchloroformate, vinylchloroformate, 1,4-butandiol-bis(chloroformate), 2-chloropropylchloroformate, propargylchloroformate or bisphenol-A-bis(chloroformate) are used.

16. The method according to claim 13, wherein as alkaline or alkaline earth compound butyl lithium, NaHCO₃ or Na₂CO₃ is used.

17. The method according to claim 13, wherein per NH-groups of the triazine 0.05 to 1.2 mol equivalents of an alkaline and alkaline earth compound, preferably 0.5 to 1.2 mol equivalents, in particular preferably 0.8 to 1.2 mol equivalents are used.

18. The method according to claim 13, wherein the reaction is carried out at temperatures of 0 to 200° C.

19. The method according to claim 13, wherein a temperature profile is being conducted during the reaction.

20. The method according to claim 19, wherein the reaction is started at low temperatures, in particular at temperatures between 0° C. and 20° C. and is then increased to a selected final temperature, in particular temperatures between 20° C. to 200° C.

21. The method according to claim 13, wherein per mol equivalent NH-groups of the triazine 0.7 to 10.0 mol, preferably 0.9 to 7.0 chloroformate are used.

22. The method according to claim 13, wherein the reaction is carried out in a solvent or in a substance, wherein the chloroformate according to formula (V) and/or (VI) acts as a solvent.
